(19) United States
(12) Patent Application Publication
(10) Pub. No.: US 2013/0192130 A1
(43) Pub. Date: Aug. 1, 2013

(54) SYSTEMS AND METHODS FOR HARVESTING AND DEWATERING ALGAE

(71) Applicant: Nicholas Eckelberry, Los Angeles, CA (US)

(72) Inventor: Nicholas Eckelberry, Los Angeles, CA (US)

(21) Appl. No.: 13/753,484

(22) Filed: Jan. 29, 2013

Related U.S. Application Data
(60) Provisional application No. 61/592,522, filed on Jan. 30, 2012.

(51) Int. Cl. A01G 7/04; A01H 13/00

(52) U.S. Cl. CPC A01G 7/04; A01H 13/00
USPC 47/1.3

(57) ABSTRACT

An applied electrical field effects the harvesting of algae from a growth medium through increased interface potential between solvent and solute and the use of micron-sized bubbles of hydrogen and oxygen gas. The process and method makes use of strategically placed bipolar electrode plates that generate hydrogen and oxygen gas. Micro bubbles of the gas flocculate the biomass out of solution concurrently clarifying the water for re-use in an algae growth system. The flocked algae can then be processed for use in applications which require a chemical-free and dewatered product such as required for bio-fuels, pharmaceuticals or food.

### CROSS-REFERENCE TO RELATED APPLICATIONS

This application claims priority to U.S. Provisional Patent Application No.: 61/592,522, filed Jan. 30, 2012, titled Systems And Methods For Harvesting And Dewatering Algae.

### BACKGROUND

There is a history of separating materials from liquid suspension in several industries, including the wastewater treatment industry and algae farming industry. Processes involved in achieving separation can vary, along with the desired end result. For example, in the wastewater treatment industry, the desired result is typically treated water that can be released into the environment. In contrast, in the algae farming industry, the primary desired result may be the harvest of a usable biomass for energy production.

There is a long history of electro-flocculation in the wastewater industry. It has been found to be an effective method of separating solids from fluids in the secondary stage of remediation. This waste stream contains organic material of all types and algae are considered a nuisance generated by the high nitrate count common in the stream. Therefore, efforts in algae eradication usually does not include preservation of the integrity of the mass for further uses such as pharmaceutical or other high value feedstock.

In electro-flocculation, as commonly used in wastewater treatment, a metal ion or cation is added to improve flocculation by increasing conductivity of the matrix. The following cations have lower electrode potential than H+ and are therefore considered suitable for use as electrolyte cations in these processes: Li+, Rb+, K+, Cs+, Ba2+, Sr2+, Ca2+, Na+and Mg2+ (sodium and lithium are frequently used as they form inexpensive salts). Other metals are used in conjunction with electro-flocculation to assist in precipitation of solids from the waste water, such as iron oxides and other oxidants. These metals are extremely effective at precipitating solids out of solution; however, they taint the product and the water itself with an inorganic chemical that then must be removed or otherwise processed in the tertiary waste treatment phase.

In practice, the current used by the wastewater systems for electro-flocculation is generally low, typically under 1 amp, as the processes are carried out in large ponds and/or in conjunction with massive fluid flows typical to a waste treatment plant which can be in the millions of gallons per day. Due to the sheer size of the plants and Ohms law (I=V/R) the current requirement and the scale of the process, it is not practical to utilize high-energy electro-flocculation systems for extended periods of time. Furthermore, the deterioration and scaling of electrolytic plates operating at high current for extended periods of time precludes the effective use of this technology at high amperage. The conductivity of the waste flow therefore must be enhanced by metal ions as discussed above to lower the energy requirement and make the process practical.

In algae product farming and harvesting, the considerations are reversed as the biomass in suspension is considered an asset whose qualities must be preserved and the use of metals taints the product irreversibly. Most methods used to dewater the algae in suspension therefore consist of centrifuge, membrane filtration, air drying with possible chemical processing and decontamination. The use of chemicals in dewatering commonly prevents or limits reuse of the growth water. The related prior application Ser. No. 13/274,094, filed Oct. 14, 2011 and titled Systems, Methods, and Apparatuses for Dewatering, Flocculating, and Harvesting Algae Cells, which is incorporated by reference discloses some forms of electro-magnetic flocculation systems. That application focuses on cell lysing as the end product.

The harvesting of microorganisms and intracellular products of microorganisms such as algae shows promise as a partial or full substitute for fossil oil derivatives or other chemicals used in manufacturing products such as pharmaceuticals, cosmetics, industrial products, biofuels, synthetic oils, animal feed, and fertilizers. However, for these substitutes to become viable, methods for harvesting the cells, including steps of recovering and processing of intracellular products must be efficient and cost-effective in order to be competitive with the refining costs associated with fossil oil derivatives. Current extraction methods used for harvesting microorganisms such as algae to ultimately yield products for use as fossil oil substitutes are laborious and yield low net energy gains, rendering them unviable for today's alternative energy demands. Such previous methods can also produce a significant carbon footprint, exacerbating global warming and other environmental issues. These prior methods, when further scaled up, produce an even greater efficiency loss due to valuable intracellular component degradation and require greater energy or chemical inputs than what is currently financially feasible from a microorganism harvest. For example, the cost per gallon for microorganism bio-fuel is currently approximately nine times the cost of fossil fuel.

All living cells, prokaryotic and eukaryotic, have a plasma transmembrane that encloses their internal contents and serves as a semi-porous barrier to the outside environment. The transmembrane acts as a boundary, holding the cell constituents together, and keeps foreign substances from entering. According to the accepted current theory known as the fluid mosaic model (S. J. Singer and G. Nicolson, 1972, incorporated herein by reference), the plasma membrane is composed of a double layer (bi-layer) of lipids, an oily or waxy substance found in all cells. Most of the lipids in the bilayer can be more precisely described as phospholipids, that is, lipids that feature a phosphate group at one end of each molecule.

Within the phospholipid bilayer of the plasma membrane, many diverse, useful proteins are embedded while other types of mineral proteins simply adhere to the surfaces of the bilayer. Some of these proteins, primarily those that are at least partially exposed on the external side of the membrane, have carbohydrates attached and therefore are referred to as glycoproteins. The positioning of the proteins along the internal plasma membrane is related in part to the organization of the filaments that comprise the cytoskeleton, which helps anchor them in place. This arrangement of proteins also involves the hydrophobic and hydrophilic regions of the cell.

Intracellular extraction methods can vary greatly depending on the type of organism involved, their desired internal component(s), and their purity levels. However, once the cell has been fractured, these useful components are released and typically suspended within a liquid medium which is used to house a living microorganism biomass, making harvesting these useful substances difficult or energy-intensive.

In most current methods of harvesting intracellular products from algae, a dewatering process has to be implemented in order to separate and harvest useful components from a liquid medium or from biomass waste (cellular mass and debris). Current processes are inefficient due to required time frames for liquid evaporation or energy inputs required for drying out a liquid medium or chemical inputs needed for a substance separation. Additionally, such processes are commonly limited to batch processing and are difficult to adapt for continuous processing systems.

Accordingly, there is a need for a simple and efficient procedure for dewatering microorganisms, such as algae, so that they can be harvested and their intracellular products can be recovered and used as competitively-priced substitutes for fossil oils and fossil oil derivatives required for manufacturing of industrial products.

### BRIEF SUMMARY

The present invention is generally directed to systems and methods for harvesting algae using a simple, low cost approach. Implementation of the invention produces a biomass that can then be lysed for oil separation while returning clarified nutrient rich water to the growth system. The cost of flocculation is reduced by using short-duration bursts of energy in strategically placed locations. These bursts of energy can be applied within the growth system or in a standalone batch processor.

A further advantage is realized by utilizing a protic polar acid within the growth and/or extraction system. The protic polar acid enhances algae growth, and, when energized by amphoteric plates, clarifies the water thereby eliminating the requirement that metal ions be used.

In one embodiment, the present invention is implemented as a system for harvesting and dewatering algae. The system includes a container capable of holding an algae solution; a cathode disposed within the container; an anode disposed within the container a distance of between approximately 1 inch and approximately 10 inches from the cathode; and a voltage source electrically connected to the cathode and the anode. The voltage source is configured to supply a voltage between the cathode and anode when the container holds the algae solution. The voltage between the cathode and anode causes bubbles of hydrogen gas to be formed in the algae solution which attach to algae cells in the algae solution causing the algae cells to float to the surface of the algae solution.

In another embodiment, the present invention is implemented as a method for harvesting and dewatering algae. The method includes supplying an algae solution to a algae dewatering apparatus. The algae dewatering apparatus comprises a container capable of holding an algae solution; a cathode disposed within the container; and an anode disposed within the container a distance of between approximately 1 inch and approximately 10 inches from the cathode. Then, a voltage is supplied between the cathode and anode to cause bubbles of hydrogen gas to be formed at the cathode and to pass through the algae solution while attaching to algae cells and floating the algae cells to the surface of the algae solution. Next, the floating algae cells are removed from the surface.

In another embodiment, the present invention is implemented as a method for harvesting and dewatering algae from a growth medium. An electric field is generated between at least one anode and at least one cathode that are submerged within a growth medium containing algae. The at least one anode and the at least one cathode are configured to generate hydrogen or oxygen bubbles within the growth medium when the electric field is generated. The hydrogen or oxygen bubbles attach to algae within the growth medium causing the algae to float to the surface of the growth medium. Then, the floating algae is removed from the surface of the growth medium.

This summary is provided to introduce a selection of concepts in a simplified form that are further described below in the Detailed Description. This Summary is not intended to identify key features or essential features of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIGS. 1A and 1B illustrate an exemplary batch process system in which algae can be dewatered according to embodiments of the invention;

FIG. 2 illustrates a harvesting system that is incorporated into a raceway;

FIGS. 3A-3D illustrate another exemplary algae harvesting system; and

FIG. 4 illustrates variations of electrode configurations that can be used in some embodiments of the invention.

### DETAILED DESCRIPTION

The present invention is generally directed to systems and methods for harvesting algae using a simple, low cost approach. Implementation of the invention produces a biomass that can then be lysed for oil separation while returning clarified nutrient rich water to the growth system. The cost of flocculation is reduced by using short-duration bursts of energy in strategically placed locations. These bursts of energy can be applied within the growth system or in a standalone batch processor.

A further advantage is realized by utilizing a protic polar acid within the growth and/or extraction system. The protic polar acid enhances algae growth, and, when energized by amphoteric plates, clarifies the water thereby eliminating the requirement that metal ions be used.

In one embodiment, the present invention is implemented as a system for harvesting and dewatering algae. The system includes a container capable of holding an algae solution; a cathode disposed within the container; an anode disposed within the container a distance of between approximately 1 inch and approximately 10 inches from the cathode; and a voltage source electrically connected to the cathode and the anode. The voltage source is configured to supply a voltage between the cathode and anode when the container holds the algae solution. The voltage between the cathode and anode causes bubbles of hydrogen gas to be formed in the algae solution which attach to algae cells in the algae solution causing the algae cells to float to the surface of the algae solution.

In another embodiment, the present invention is implemented as a method for harvesting and dewatering algae. The method includes supplying an algae solution to a algae dewatering apparatus. The algae dewatering apparatus comprises a container capable of holding an algae solution; a cathode disposed within the container; and an anode disposed within the container a distance of between approximately 1 inch and approximately 10 inches from the cathode. Then, a voltage is supplied between the cathode and anode to cause bubbles of hydrogen gas to be formed at the cathode and to pass through the algae solution while attaching to algae cells and floating the algae cells to the surface of the algae solution. Next, the floating algae cells are removed from the surface.

In another embodiment, the present invention is implemented as a method for harvesting and dewatering algae from a growth medium. An electric field is generated between at least one anode and at least one cathode that are submerged within a growth medium containing algae. The at least one anode and the at least one cathode are configured to generate hydrogen or oxygen bubbles within the growth medium when the electric field is generated. The hydrogen or oxygen bubbles attach to algae within the growth medium causing the algae to float to the surface of the growth medium. Then, the floating algae is removed from the surface of the growth medium.

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

Unless otherwise defined, all technical terms used herein have the same meaning as commonly understood by one of ordinary skill in the art to which embodiments of the invention belong.

According to embodiments of the invention, an algae growth medium is flocculated in a manner that causes the algae cells to float to the surface where they can easily be extracted (e.g. using a rake). An electrical field can be applied to the growth medium using electrodes. The electric field increases interface potential between solvent and solute and creates micron-sized bubbles of hydrogen and oxygen gas which lift the flocked algae to the surface.

The process and method makes use of strategically placed bipolar electrode plates that generate hydrogen and oxygen gas. Micro bubbles of the gas flocculate the biomass out of solution concurrently clarifying the water for re-use in an algae growth system. The flocked algae can then be processed for use in applications which require a chemical-free and dewatered product such as required for bio-fuels, pharmaceuticals or food.

Certain embodiments of the invention utilize introduction of protic solvents, such as formic acid, n-butanol, isopropanol, n-propanol, ethanol, methanol, and acetic acid, which are considered benign to an overall algae growth system. Additionally, electrodes, such as electrolytic plates and/or rods, may be used that are also benign to the algae growth system.

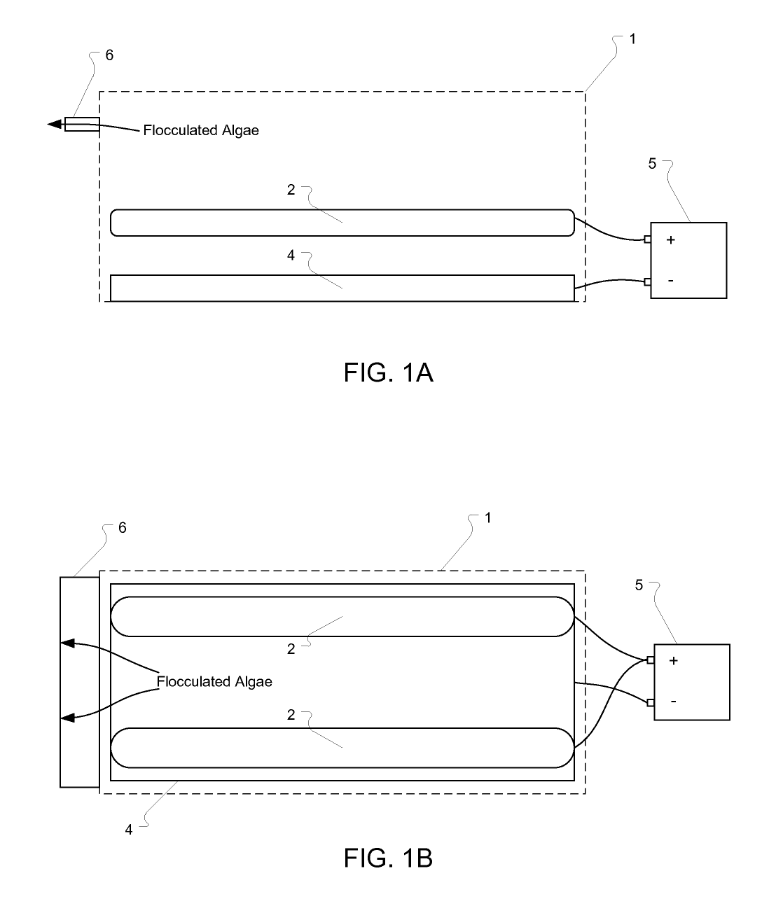

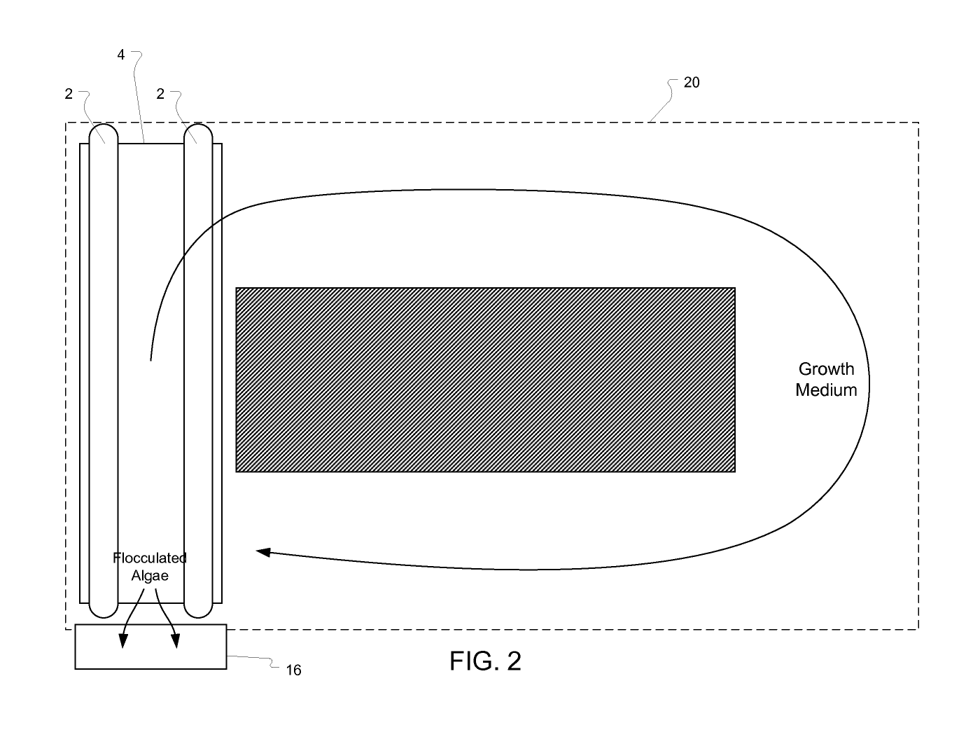

In one embodiment, such as shown in FIGS. 1A and 1B, a batch process system is utilized, wherein algae stock, grown to maturity, is processed through an enclosure where the electro-flocculation process occurs to substantially-complete flocculation of the product. FIG. 1A illustrates a side view while FIG. 1B illustrates a top view of a tank 1.

The growth medium is enclosed in tank 1 where it is brought into contact with the bottom electrode plate 4 and the two top bipolar electrode tubes 2. A battery 5 applies a voltage differential (which may be reversed from what is shown in the figures) between electrode plate 4 and electrode tubes 2 thereby causing a current to flow through the growth medium. This current causes the growth medium to flocculate.

In other flocculation techniques, it is often difficult to separate the flocculated algae cells from the growth medium and generally requires the use of chemicals or techniques that make the algae cells unsuitable for many uses. However, the present invention uses small bubbles of hydrogen and oxygen to attach to clumps of algae cells and lift the clumps to the surface of the growth medium. Once at the surface, the flocculated product is then discharged through a plate 6 for further processing. Although a single plate 4 and two tubes 2 are shown, different numbers of plates 4 and/or tubes 2 can be used.

Additionally, in some embodiments, the growth medium may be injected with a dilute solution of a protic solvent such as formic acid, n-butanol, isopropanol, n-propanol, ethanol, methanol, and acetic acid, such as of approximately 0.05% by volume. This solution may be mixed into the matrix as the electric field of the electro-flocculation process is generated, or just before the batch process occurs. The voltage used in tests to date is substantially constant at approximately 12 volts; however, the amperage may vary from approximately 5 amps to approximately 10 amps depending on the density of the feedstock and its effect on Ohm's law.

The plate(s) 4, electrode tube(s) 2, or other electrodes should be composed of relatively inert and benign metals which do not impart contamination to the end product or the growth water, thereby permitting reuse of the growth water. Although non-benign metals may be used where reuse of the growth water and/or contamination of the collected product is not a concern. To permit reuse of the growth water, metals such as copper cannot be used as they are algaecides. Stainless steel in time would deteriorate and impart chromium to the growth water. Therefore, carbon, aluminum, and platinum group metals are generally recognized as safe and preferred for this process.

Testing has shown that the apparatus is capable of flocculation of the algae stock of 350 mg/l to complete clarity of water with the algae in matted form on the surface in 1.50 minutes with 12v DC and 9.5 amps constant current. In the experiment only one aluminum rod was used as an anode on top and the length of the tank.

The distance between the lower plate 4 and the upper tube(s) 2 can vary between approximately 1 inch and approximately 10 inches so as to permit the generated oxygen and hydrogen bubbles to flow freely upwards. The generation of micro bubbles of oxygen on the anode tube 2 and micro bubbles of hydrogen on the cathode plate 4 creates the bubbles necessary for flocculation.

As discussed above, FIG. 1 shows an embodiment adapted for batch flocculation of algae from a growth medium. The batch process apparatus of FIG. 1 may be operatively coupled to an algae growth system, and batches of growth medium may be transferred from the algae growth system to the batch process apparatus of FIG. 1 from time to time as sufficient growth of the algae has occurred. As discussed above, the growth medium remaining after electro-flocculation (which may be nutrient dense) may be returned to the algae growth system for reuse.

Alternatively, embodiments of the invention may be directly incorporated into algae growth systems, such that the electro-flocculation process may occur within the growth system, either intermittently as needed or even continuously. FIG. 2 illustrates such a configuration. The system of FIG. 2 includes a raceway 20 where algae is grown and nourished in a scheduled manner. In this embodiment, the growth medium flows in a clockwise manner and is electro-flocked with a DC generator (not shown), the bottom electrode plate 4, and the bipolar electrode tube(s) 2 as the growth medium progresses through the portion of raceway 20 containing the elctro-flocculation apparatus. Biomass is collected through a weir 16.

Thus, when features of the invention are embodied in a continuous growth system bio-mass extraction system, the apparatus is positioned within the fluid flow of a pond, raceway or other growth system and is activated manually or automatically (e.g. by a distributed control system that adjudicates through metrics such as pH, ORP, density, colorimeter reading or cell count among others), thus achieving the proper dispensation of current at timed intervals to extract mature cells while retaining the integrity of the overall matrix.

Regardless of whether the embodiment of the invention is a batch process embodiment as illustrated in FIG. 1 or an intermittent to continuous process embodiment as illustrated in FIG. 2, it has been found that inoculating the growth medium with a benign protic solvent in minute proportions during the growth phase has benefit in enhancing the growth cycle. The presence of the protic solvent in the growth medium at the time of electro-flocculation additionally serves as a clarifying agent that facilitates the separation of the algae from the growth medium. This may be due to improved formation of micro bubbles of hydrogen gas at the cathode when the protic solvent is present.

In the use of these systems, a plurality of hydrogen and oxygen gases is generated as a consequence of the hydrolysis process. The gas appends itself to the alga cells and entrains them to the top of the growth medium, effectively decreasing the otherwise-neutral density of the algal cells. In this form, the gases and algae form a mat which becomes an intrinsic part of the floc. While one can utilize the floc in this composition with hydrogen (and/or oxygen) gas present in the floc, a recovery system for this high value gas can be designed and utilized for energy mitigation or other usage, thereby recapturing a portion of the input energy used in hydrolysis.

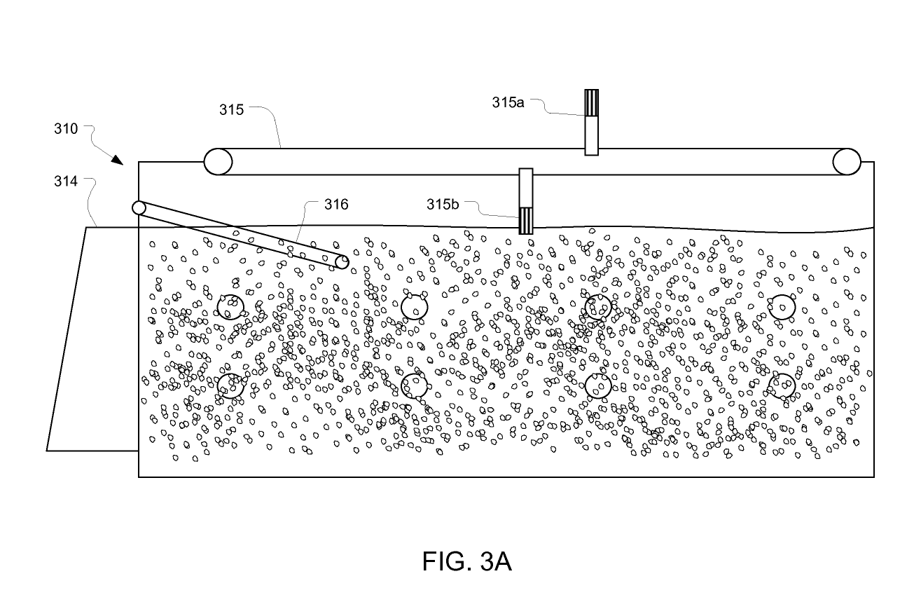

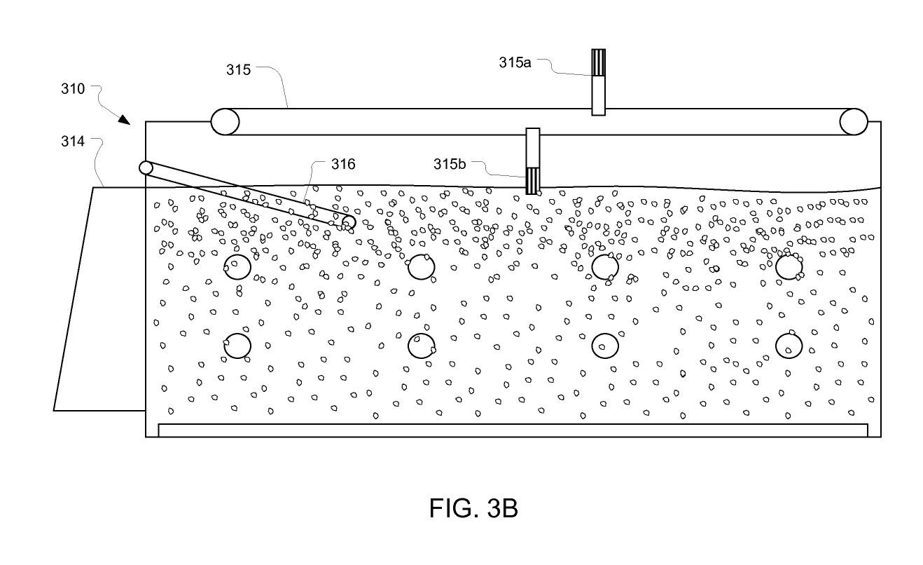

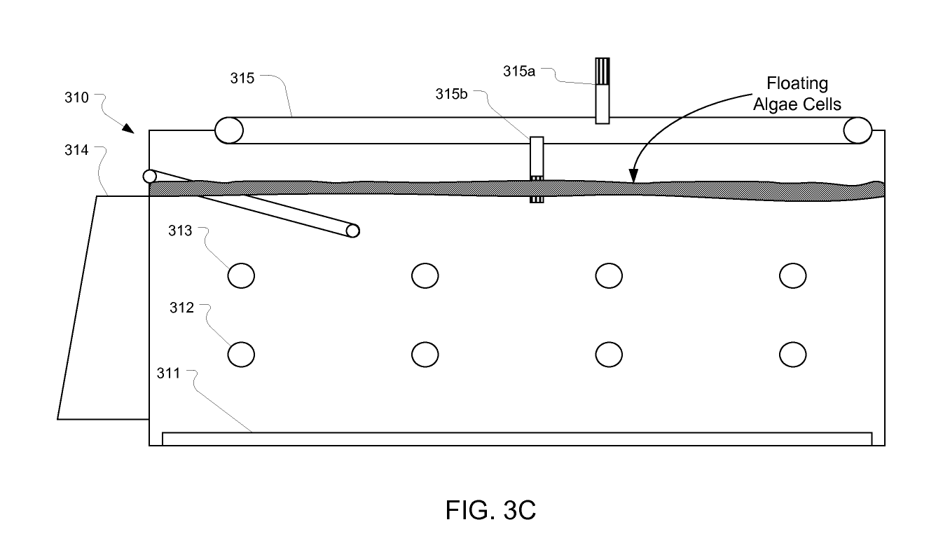

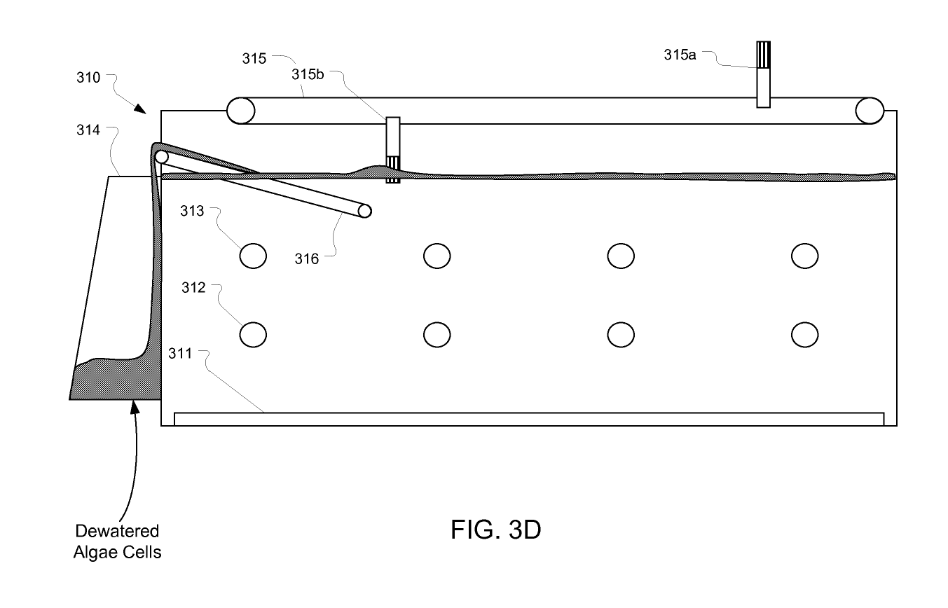

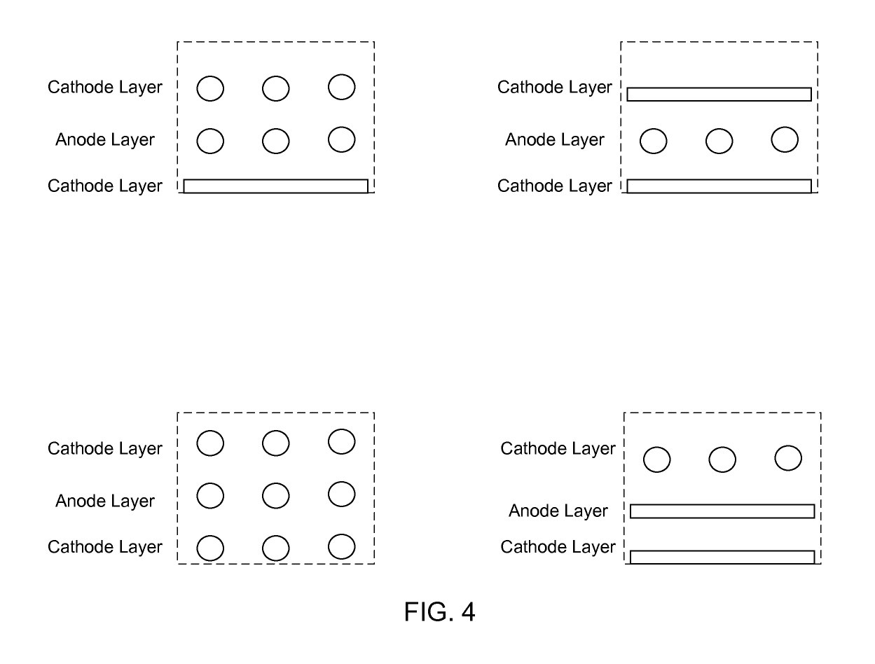

FIGS. 3A-3D illustrate an exemplary container 310 in which the algae harvesting techniques of the present invention can be implemented. Container 310 includes a cathode plate 311 and a series of stacked anode 312 and cathode 313 rods. However, cathode rods 313 are not required as is shown in FIGS. 1 and 2. Other configurations of electrodes can also be used within container 310 as shown in FIG. 4. Container 310 also includes conveyor 315 (having rakes 315a and 315b) and conveyor 316 which are used to remove the algae cells from container 310 and into collector 314 as will be further described below. Other means for removing the algae from the surface of the growth medium can also be used as in known in the art.

FIG. 3A illustrates the state of container 310 once flocculated algae cells exist within the growth medium. In some embodiments, a growth medium that already contains flocculated algae cells can be introduced into container 310. In other embodiments, a growth medium having various concentrations of algae cells can be introduced into container 310. For example, a growth medium containing algae ready for flocculation can be introduced as in FIG. 1, or a growth medium requiring additional algae growth before flocculation can be introduced as in FIG. 2.

As stated above, prior approaches for separating algae from the growth medium are difficult, expensive, and oftentimes harmful to the algae making them unsuitable to recover algae that is intended for certain purposes. In contrast, the present invention provides a simple and safe process for recovering the algae cells. This process includes applying an electric field to the growth medium using electrodes 311, 312, and, in some cases, 313. In some cases, this electric field can cause the algae cells in the growth medium to flocculate into clumps as shown in FIG. 3A (e.g. if the clumps have not already been formed prior to the growth medium being introduced into container 310, or by growing the size of the clumps). In some embodiments, the clumps can be between 1 and 4 mm.

In addition to forming clumps, the electrodes can be configured to cause hydrogen and oxygen gas bubbles to form which adhere to the clumps and lift them to the surface as shown in FIG. 3B. In some embodiments, a protic solvent can also be added to the growth medium to enhance the flocculation of the algae cells and to enhance the separation of the flocculated algae from the growth medium.

FIG. 3C illustrates the state of container 310 after the clumps of algae cells have floated to the surface. FIG. 3C also illustrates that the remaining growth medium underneath the floating clumps is substantially clear to indicate that this process is highly effective at separating the algae from the growth medium. The growth medium, which is nutrient dense, can then be reused.

Finally, FIG. 3D illustrates an example of how the floating algae cells can be removed. As shown, this removal can be performed using rakes 315a, 315b which are rotated over the surface of the growth medium to rake the algae cells towards conveyor 316. Conveyor 316 is rotated to transfer the raked algae cells into collector 314 where it can be retrieved for further processing. Accordingly, this process yields a highly dewatered biomass that can be easily transported and used.

FIGS. 3A-3D generally represent the process as being performed in batches (i.e. the entire growth medium is fully flocculated before any new algae cells are added). However, in some embodiments, this process can be performed on a continuous basis such as by periodically adding new growth media containing algae (whether flocculated to some degree or not).

FIG. 4 illustrates alternative arrangements of the electrodes that can be used in some embodiment of the invention. As shown, the electrodes can be arranged in a three layer configuration. Other configurations can also be used. Also, different spacings between layers can be used.

Experimental Results

The following tests were conducted to evaluate the principles set forth in the embodiments of the invention discussed above. The tests results are included to illustrate the principles discussed herein and are not intended to be limiting to the scope of the invention. Additionally, in some of the tests, a copper (Cu) electrode was used, but it should be understood in light of the discussion above that copper electrodes would not normally be used where growth medium is to be reused due to the algaecidal property of copper.

Test 1

The test was to determine the value of the MX (transient cavitation generator and mixer as disclosed in U.S. Pat. No. 6,279,611, incorporated herein by reference) in flocculation, cell cracking, and other ancillary benefits if any.

After a few trial and error runs, the test was configured as follows: 1) MX injection of Kalkwasser, (a filtered Calcium Hydroxide water blend), 2) Application of electrical current to crack the algae, 3) Recovery of product (floc), and 4) Analyses at three different stages for evidence of cell lysing.

The most interesting result was attained when the following protocol was followed: 1) A slurry of 1 liter high pH (11.4) kalkwasser was injected in 5 liters of good density (app: 500 mg/l) nannocholoropsis algae in salt water with the MX and circulated for 1 minute at micron stage. 2) The resulting product was an increase of roughly 1 pH point (8.4 to 9.2) within <1 minute. 3) two rods: aluminum and copper were placed at either side of the tank and ran the whole length of the tank hooked up to a voltage generator, 4) An electrical charge was applied for 2 minutes at +/−6. 0 Volt and 3.25 amps.

The visible result was thorough cell lysing and the formation of what appeared to be hydrophobic drop on the slide. While this drop looks like oil and reacted like oil, it would be a stretch to conclude that it was a lipid of any type. The cells were, however, destroyed quite convincingly as recorded on three micrographs.

Observations: The application of electricity via two rods (anode and cathode) in water was effective so long as the rods were separated by the full width of the tank. Narrowing the gap lowered the amperage and voltage to no result or extreme increased time to lysing. There appeared to be a correlation in some of the tests to m/s (micro-Siemens) increasing as the product was lysed: this is yet to be fully confirmed, however might prove to be a possible metric to determine lysing

The use of a high pH prior to electrification appeared to assist in the lysing process. The use of high pH prior to lysing appeared to create a dramatic floc in a shorter period of time. The use of Micron-bubble mixing enhanced the process of pH management by making a near instant adjustment to the whole matrix. This would have value in large scale operations.

The use of rods in a tank for electro-flocculation and cell lysing had value with a low voltage (6 volt and 3.25 amps) for a short period (approximately 2 minutes). The MX was run continuously during this electro-stage to disperse the matrix widely; the matrix contained a very large amount of hydrogen that was peeling off the cathode. Other tests will be done with and without this agitation to determine the value of the micronization step in electro-flocculation/lysing. It should be noted that the cathode was on the aluminum rod versus the copper rod so as to mitigate the production of Cu2O. (The cathode is used in this case to decompose biological material and was plugged into the negative side of the voltage generator and the positive to Cu.) Al20 (alumina) is much harder to produce at this electrical level, and much less degradation of the cathode was noted than would have occurred with a copper cathode.

Test 2

Protocol: A tank was set up with an anode plate placed on the bottom of the tank. The cathode strip (Al) was set up approximately 4 inches from the anode (Cu). Both the anode and the cathode ran the length of the tank. Low-density algae solution (approximately 200 mg/l) was dumped into the tank just above the cathode plate and the product was subjected to MX circulation. Concurrent with pump-driven micro-bubbling of the product a low voltage was applied: 4 volts and 3.25 amps. The whole mixture was run for a period of two minutes, when all processes were halted.

Visual Results: The whole of the bio-mass rapidly floated to the top and flocculated within 1 minute or so. The mass appeared green with no visible discoloration. The mass could easily be skimmed off at this point. A micrograph was pulled and showed large scale cellular distention and evidence of cracking The bulk of the mass stayed at the top of the tank for 24 hours, indicating the possibility of a high concentration of lighter than water product.

Conclusions: The concept of two plates that run the length of the tank and are separated by many inches has validity. The concept of a large scale “cracking tank” is possible with little infrastructure costs and low operational energy costs. The micro-bubbles appeared to assist in the flocculation of the bio-mass as the hydrogen, oxygen and bio-mass was subjected to intense mixing. When the micro-bubbles pump was halted the bubbles rose to the top, thereby enhancing the process.

The design of a large scale fast flocking and cracking tank is possible through this methodology. It should be noted that no chemicals were used to induce flocculation. The remaining water therefore can go back into circulation with no more handling or clean up. It should also be noted that a large quantity of hydrogen was produced at the top of the tank, which could be harvested. This process defies the current methodology of close proximity of plates and also provides for a “floating cathode” which places it directly at the top of the matrix whereby it continuously provides positive current to the flocculated bio-mass.

Test 3

Protocol: To study the difference between MX usage and no MX usage in the floc/cracking system. Two identical amounts of product were processed. The density appeared to be roughly 400 mg/l good product.

The first lot was electrolyzed with 2 plates. The initial voltage was 3.0 and 3.25 amps. After 11 minutes, the mass started flocculating and the voltage jumped to 4.5 Volts. Application of the voltage ceased and micro-examination of the now flocculated mass performed. It showed cracking and visible exuded material from cells.

The second lot was processed with MX for 2 minutes with electricity; the initial voltage was 3.0 V. and 3.25 amps. The MX was stoped and the electrolyzing process continued for 3 minutes and attained the same jump in Voltage to 4.5. The examination of biomass showed extensive cracking and the mass was completely flocked to the top.

Conclusions: 1) The cell cracking and floc appears to correlate with a voltage increase, permitting the possibility of a metric to determine when the cell is cracked. 2) It appears that the MX halves the time to product. Whether this is an advantage or not needs to be determined by costs analyses and biomass quality. 3) This method of cracking algae is very fast, efficient and shows great promise as a method of lysing and flocculating. 4) The bio-mass in this case was much higher density: app: 400 mg/l. and the process worked well and efficiently.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. A system for harvesting and dewatering algae comprising:
a container capable of holding an algae solution;
a cathode disposed within the container;
an anode disposed within the container a distance of between approximately 1 inch and approximately 10 inches from the cathode;
a voltage source electrically connected to the cathode and the anode and configured to supply a voltage between the cathode and anode when the container holds the algae solution, wherein the voltage between the cathode and anode cause bubbles of hydrogen gas to be formed in the algae solution which attach to algae cells in the algae solution causing the algae cells to float to the surface of the algae solution.

2. A system as recited in claim 1, wherein the cathode is a plate and the anode is one of a plurality of anode rods, each disposed a distance of between approximately 1 inch and approximately 10 inches above the cathode plate.

3. A system as recited in claim 2, further comprising:
a plurality of cathode rods, each disposed a distance of between 1 inch and 10 inches above an anode rod.

4. A system as recited in claim 2, further comprising:
a second cathode plate positioned between 1 inch and 10 inches above the anode rods.

5. A system as recited in claim 1, wherein the cathode and anode are plates.

6. A system as recited in claim 5, further comprising:
a plurality of cathode rods, each disposed a distance of between 1 inch and 10 inches above the anode plate.

7. A system as recited in claim 1, wherein the cathode comprises a plurality of cathode rods, and the anode comprises a plurality of anode rods, each disposed a distance of between approximately 1 inch and approximately 10 inches above a corresponding cathode rod, the system further comprising:
a second plurality of cathode rods each disposed a distance of between 1 inch and 10 inches above a corresponding anode rod.

8. A system as recited in claim 1, wherein the system is incorporated into a raceway.

9. A system as recited in claim 1, wherein one or more of the anode or cathode comprises titanium coated with iridium oxide.

10. A system as recited in claim 1, wherein the container is a trench.

11. A method for harvesting and dewatering algae, comprising:
supplying an algae solution to a algae dewatering apparatus, the algae dewatering apparatus comprising:
a container capable of holding an algae solution;
a cathode disposed within the container; and
an anode disposed within the container a distance of between approximately 1 inch and approximately 10 inches from the cathode;
supplying a voltage between the cathode and anode to cause bubbles of hydrogen gas to be formed at the cathode and to pass through the algae solution while attaching to algae cells and floating the algae cells to the surface of the algae solution; and
removing the floating algae cells from the surface.

12. A method as recited in claim 11, further comprising:
adding a protic solvent to the algae solution.

13. A method as recited in claim 12, wherein the protic solvent comprises one of formic acid, n-butanol, isopropanol, n-propanol, ethanol, methanol, or acetic acid to the algae solution.

14. A method as recited in claim 13, wherein the one of formic acid, n-butanol, isopropanol, n-propanol, ethanol, methanol, and acetic acid is added at a concentration of approximately 0.05% by volume.

15. A method for harvesting and dewatering algae from a growth medium, the method comprising:
generating an electric field between at least one anode and at least one cathode that are submerged within a growth medium containing algae, the at least one anode and the at least one cathode being configured to generate hydrogen or oxygen bubbles within the growth medium when the electric field is generated, the hydrogen or oxygen bubbles attaching to algae within the growth medium causing the algae to float to the surface of the growth medium; and
removing the floating algae from the surface of the growth medium.

16. The method of claim 15, wherein the at least one anode and the at least one cathode comprise a cathode plate positioned below one or more anode rods.

17. The method of claim 16, wherein the at least one anode and the at least one cathode comprise a three layer stack including a bottom cathode layer, a middle anode layer, and a top cathode layer.

18. The method of claim 17, wherein the bottom cathode layer comprises a plate, and the middle anode layer comprises a plurality of anode rods.

19. The method of claim 18, wherein the growth medium contains acetic acid to enhance the separation of the algae from the growth medium.

20. The method of claim 15, wherein the electric field causes the algae to flocculate into clumps to which the hydrogen or oxygen bubbles attach thereby floating the clumps to the surface.
